(19) United States
(12) Patent Application Publication
(10) Pub. No.: US 2012/0071716 A1
(43) Pub. Date: Mar. 22, 2012

(54) MEDICAL INSTRUMENT WITH MULTIPLE FUNCTIONS FOR AN ENDOSCOPE

(75) Inventors: Olivier Fructus, Nazelles Negron (FR); Nicolas Mathieu, Ecully (FR)

(73) Assignee: AXESS VISION TECHNOLOGY, Tours (FR)

(21) Appl. No.: 13/321,239

(22) PCT Filed: May 19, 2010
(86) PCT No.: PCT/FR10/50976

§ 371 Date: Nov. 18, 2011

(30) Foreign Application Priority Data
May 19, 2009 (FR) 0953308

(51) Int. Cl. A61B 1/018

(52) U.S. Cl. 600/106

(57) ABSTRACT

The invention relates to a medical instrument for an endoscope, the instrument comprising a flexible outer insertion tube (5) having a tubular wall possessing a proximal portion (6) for connection to an actuator support (3) and a distal portion (7) presenting an outlet section (8) and connected to a folding mechanism. The instrument comprises:
an outer insertion tube (5) includes closure systems (10, 11) for closing its proximal portion (6) and its distal portion (7) to define an internal chamber (13) communicating beside the distal portion with the outside of the tube via at least one orifice (15) formed through the distal portion of the tubular wall, and beside the proximal portion, with a substance transit source (19); and
a series of flexible tubular ducts (21ᵢ) mounted independently of one another inside the insertion tube (5) passing through the chamber (13) and the closure systems (10, 11) in order to open out to the outside of the outlet section (8) of the distal portion (7) of the tube.

The present invention relates to the technical field of instrumentation involving medical appliances in the general sense, for enabling access to be obtained to the inside of a body in the general sense such as a cavity or a channel, and suitable for being guided or steered inside said body.

More precisely, but not exclusively, the present invention relates to a single use or reusable instrument for a medical endoscope enabling access to be obtained to the inside surface of a hollow organ, a cavity, or a natural or artificial duct of the human body in order to perform various operations for therapeutic, surgical, or diagnostic purposes.

The instrument of the invention is used for diagnostic or surgical purposes for inspecting any internal portion of the human body that is accessible via a natural path or an artificial path. For example, the endoscope instrument of the invention may be used in the fields of the urinary tracts, gastrointestinal tracts, the respiratory system, the cardiovascular system, the trachea, the sinus cavity, the female reproductive system, the abdominal cavity, or any other portion of the human body that can be investigated via a natural or artificial path.

In the above technical field, there are various types of endoscope that are adapted to organs that are accessible for observation. In general, an endoscope comprises a more or less flexible insertion tube possessing a proximal portion for connection to an actuator support that serves to steer the tube inside the insertion path. The endoscope generally includes a generally optical display system making it possible from the distal portion of the insertion tube to illuminate and examine the organ, cavity, or duct in the human body.

In numerous applications, it is found to be advantageous to have one or more appliances at the distal portion of the insertion tube that are suitable for performing various functions such as, delivering fluid, sucking up fluid, delivering instruments, taking samples, or performing surgical actions. The distal portion of the insertion tube thus presents an outlet section that is occupied by a plurality of appliances. In other words, in order to carry out successfully a therapeutic, surgical, or diagnostic action, there is a need to have an endoscope instrument available that is provided with a plurality of appliances that can be used in succession or simultaneously.

This applies in particular with U.S. Pat. No. 5,483,951, which describes an endoscope having a flexible tube with the display system mounted therein, while the working channels such as channels for delivering air, delivering water, and taking samples are fitted on the outside of the flexible tube, inside a flexible sheath. It is clear that such an endoscope presents a cross-section that is relatively large.

Similarly, in the field of rigid endoscopes, and in particular a hysteroscope, document WO 2005/037088 discloses making a medical instrument comprising an outer jacket mounted on a rigid inner body having various channels arranged therein to deliver appliances. The outer jacket co-operates with the rigid body to define a fluid flow chamber communicating with the outside of the jacket via orifices formed in said jacket. In addition to the fact that such an instrument is rigid, implementing an inner body adapted to allowing multiple appliances to be delivered necessarily leads to making an inner body that is voluminous, and consequently an endoscope that presents a cross-section that is relatively large.

Furthermore, even though the miniaturization of appliances is constantly advancing, the development of appliances that are ever more complex makes it necessary to have a through section in the distal portion of the insertion tube that is relatively large.

However, it should be understood that the outlet section of the distal portion of the insertion tube presents a value that is limited by the smallest width of the natural or artificial access path into which the insertion tube is engaged.

The present invention thus seeks to remedy this contradiction relating to the need to have an instrument for an endoscope that includes a plurality of functions while also presenting size that is limited so as to enable it to pass along an access path of small diameter.

The present invention thus seeks to provide a medical endoscope instrument, in the general sense, presenting a distal portion that is miniaturized while still making a variety of functions or appliances available that may be implemented successively or simultaneously.

To achieve such an object, the medical instrument for an endoscope comprises a flexible outer insertion tube having a tubular wall possessing a proximal portion for connection to an actuator support and a distal portion presenting an outlet section and connected to a folding mechanism.

According to the invention:
the outer insertion tube includes closure systems for closing its proximal portion and its distal portion to define an internal chamber communicating beside the distal portion with the outside of the tube via at least one orifice formed through the distal portion of the tubular wall, and beside the proximal portion, with a substance transit source; and
a series of flexible tubular ducts mounted independently of one another inside the insertion tube passing through the chamber and the closure systems in order to open out to the outside of the outlet section of the distal portion of the tube.

According to a preferred embodiment characteristic, the systems for closing the distal and proximal portions are sealed for passing the tubular duct(s) in such a manner that the chamber and the tubular duct(s) are sealed relative to one another.

Advantageously, at least one tubular duct defines a through channel for passing an appliance.

For example, at least one tubular duct includes as an appliance, a substance associated with an activation system connected to the actuator support.

In a variant embodiment, at least one tubular duct is provided in the outlet section of the tube with a tearable or ejectable sealing member.

In another embodiment of the invention, the instrument includes an enclosure that is sealed relative to the chamber and that communicates beside the distal portion with the outside of the tube via at least one additional orifice formed in the distal portion of the tubular wall, and beside the proximal portion with a passage communicating with an additional substance transit source.

Advantageously, the instrument includes at least one sealing membrane closing the orifice(s) formed in the distal portion of the tubular wall, said sealing membrane being tearable by means of fluid pressure or suction or by means of mechanical traction or pressure.

The invention also proposes an endoscope including a medical instrument in accordance with the invention.

Various other characteristics appear from the following description made with reference to the accompanying drawings which show embodiments of the invention as non-limiting examples.

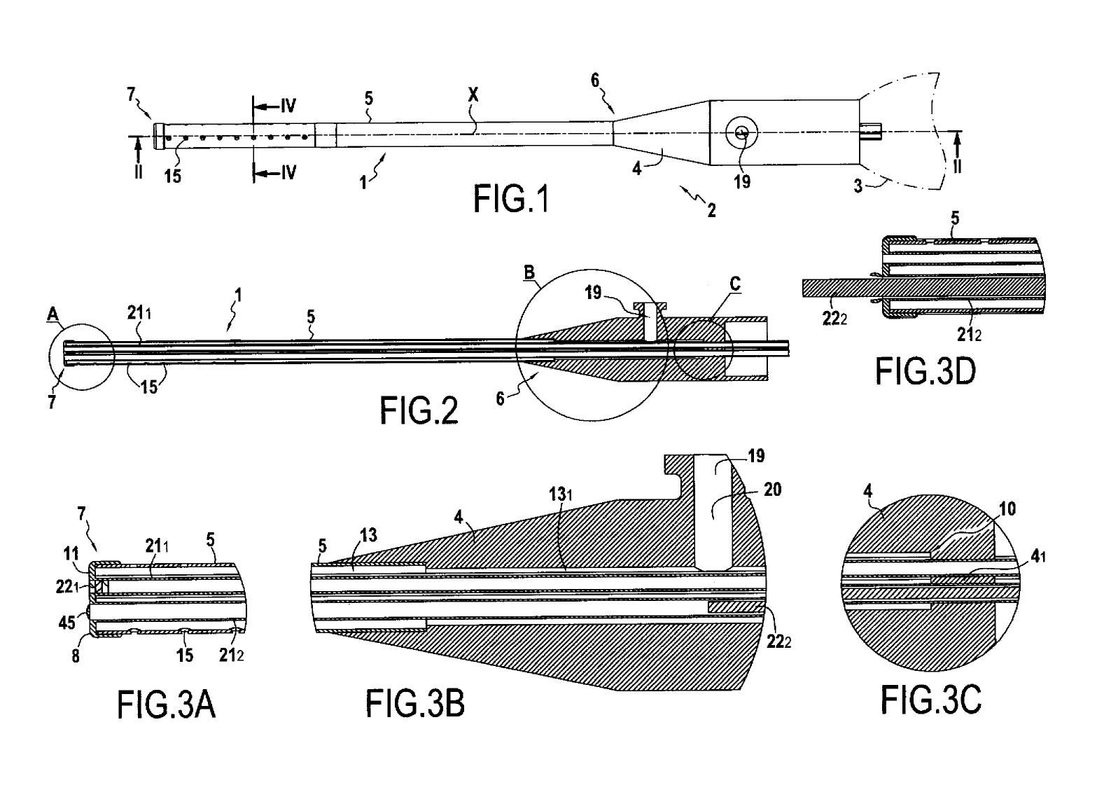

FIG. 1 is an elevation view of an embodiment of a multiple-function instrument in accordance with the invention.

FIG. 2 is a longitudinal section view substantially on line II-II of FIG. 1.

FIGS. 3A, 3B, and 3C are views on a larger scale showing respective characteristic details A, B, and C of the instrument shown in FIG. 2.

FIG. 3D is a view analogous to FIG. 3A but showing the appliances in another characteristic position.

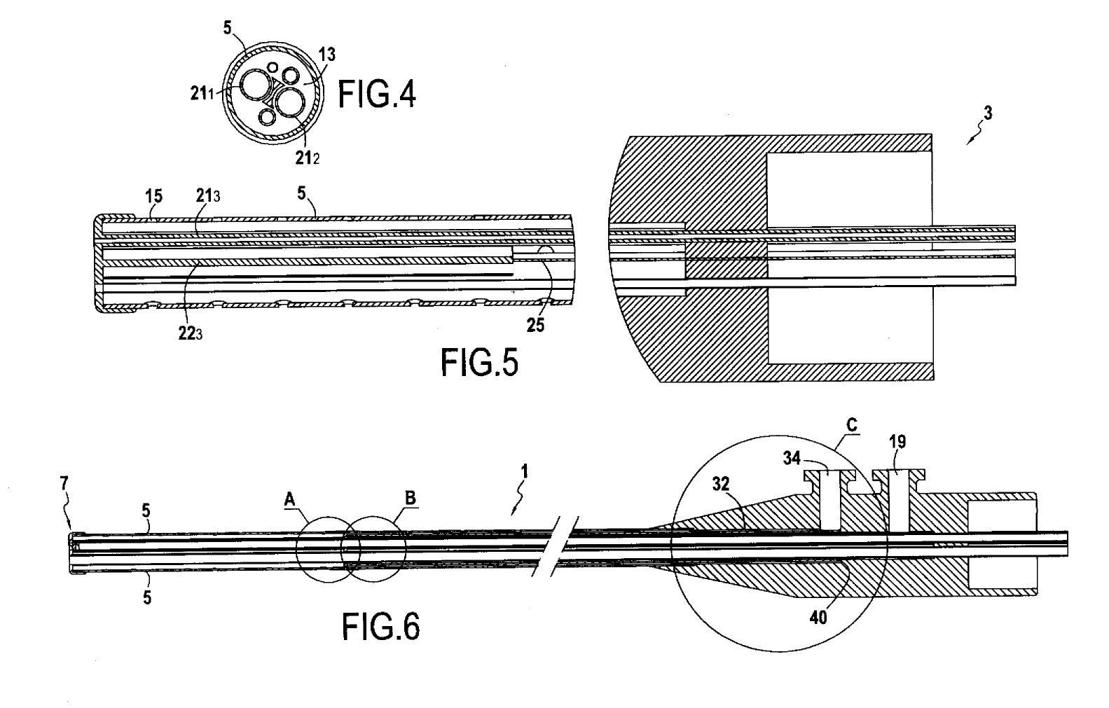

FIG. 4 is a cross-section view taken substantially on line IV-IV of FIG. 1.

FIG. 5 is a fragmentary section view showing a particular embodiment of the instrument in accordance with the invention.

FIG. 6 is a longitudinal section view showing another embodiment of the instrument in accordance with the invention.

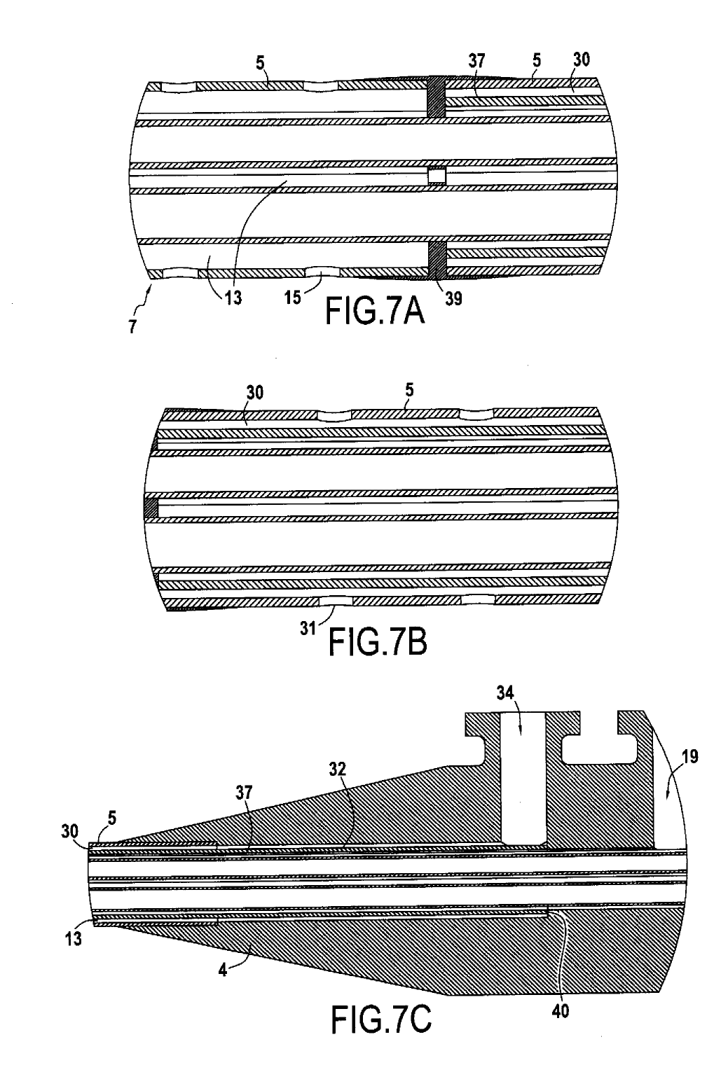

FIGS. 7A, 7B, and 7C are section views on a larger scale showing respective details A, B, and C of the instrument shown in FIG. 6.

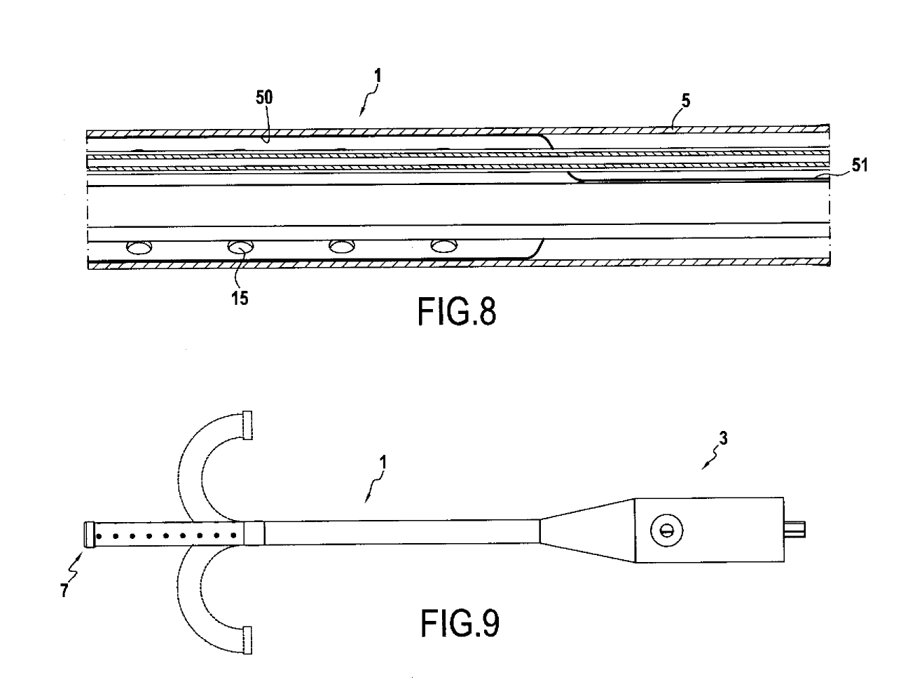

FIG. 8 is a fragmentary longitudinal section view showing another particular embodiment of the instrument in accordance with the invention.

FIG. 9 shows the instrument in accordance with the invention being folded.

As can be seen more precisely in FIG. 1, the subject matter of the invention relates to an instrument for a medical endoscope 2 that gives access to any portion of the human body, through a natural or artificial access path in order to perform various operations or functions for therapeutic, surgical, or diagnostic purposes. The endoscope 2 thus gives access to the inside surface of a hollow organ, a cavity, or a duct in the human body. It should be understood that the instrument 1 is adapted to be fitted to various types of endoscope that are adapted to organs that are accessible via a natural path such as a laryngoscope, a bronchoscope, an esophagoscope, a gastroscope, a duodenoscope, a cystoscope, a hysteroscope, and a coloscope, for example. Naturally, the instrument 1 is adapted to obtain access to various internal portions of the human body also via paths that are made artificially.

In conventional manner, a medical endoscope 2 comprises an actuator block or support 3 that is generally in the form of a handle or a robot arm, and that is fitted with the instrument 1 in accordance with the invention. In the example shown, the instrument 1 is provided with a connection endpiece 4 for connection to the actuation support 3, which support is shown diagrammatically and in part only. The instrument 1 is mounted so as to be integral with the actuator support 3, or preferably so as to be separable from the actuator support 3. In this particular example, relating more specifically to an instrument for single use, the endoscope 2 includes between the instrument 1 and the actuator block 3, a connection and disconnection system adapted to provide at least a temporary mechanical connection quickly, while also providing the advantage of enabling the instrument 1 to be separated easily from the support 3. The connection and disconnection system is not described insofar as it is well known to the person skilled in the art and does not specifically form a part of the invention.

As can be seen more clearly in FIGS. 1 to 4, the instrument 1 comprises an outer insertion tube 5 having a tubular wall extending along a longitudinal axis X and a right cross-section that is preferably circular. The outer insertion tube 5 is flexible or supple to a greater or lesser extent. The outer insertion tube 5 possesses a proximal portion 6 mounted in the example shown on the endpiece 4 for connection to the actuator support 3. The outer insertion tube 5 has a distal portion 7 remote from the proximal portion 6 and forming the head of the instrument 1. As can be seen more precisely in FIG. 3A, the distal portion 7 of the instrument 1 presents a transverse face 8 at its free end, which face defines the outlet section of the outer insertion tube 5.

The outer insertion tube 5 has a proximal system 10 for closing the proximal portion 6 of the outer insertion tube 5, and a distal system 11 for closing the distal portion 7 of the outer insertion tube 5 in such a manner that the outer insertion tube 5 internally defines a chamber 13.

In the example shown, the proximal closure system 10 is provided by a transverse wall 4₁ of the endpiece 4 (FIG. 3C). It should be observed that in the embodiment shown, the transverse wall 4₁ does not directly close the outlet section of the proximal portion 6 of the outer insertion tube 5. As can be seen more precisely in FIGS. 3B and 3C, the transverse wall 4₁ extends at a distance from the proximal portion 6 of the outer insertion tube 5. The outer insertion tube 5 is mounted in a blind bore 13₁ formed in the endpiece 4 and having its end wall provided by the transverse wall 4₁, extending at a distance from the proximal portion 6. In this example, it may be considered that the blind hole 13₁ defines a portion of the chamber 13. Naturally, it could be envisaged that the proximal portion 6 comes into abutment against the transverse wall 4₁ such that the outer insertion tube 5 is closed directly. Under such circumstances, the chamber 13 is defined exclusively by the outer insertion tube 5.

The distal closure system 11 is constituted in the example shown by a plug that closes the outlet section 8 of the outer insertion tube 5. This closure plug 11 is formed integrally with the insertion tube 5, or else, as shown, it is fitted thereto. Naturally, the proximal and distal closure systems 10 and 11 may be made differently.

Beside the distal portion 7, the chamber 13 communicates with the outside of the tube 5 via at least one orifice 15, and in the example shown via a series of orifices 15, which orifices are formed in the distal portion of the tubular wall of the outer tube 5. In the example shown, the orifices 15 are distributed over the entire circumference of the outer tube 5 and over a limited length of the distal portion 7. Naturally, the number, the shape, and the positioning of the orifices 15 are selected as a function of the intended application, as explained below in the description.

The chamber 13 communicates beside the proximal portion 6 with a transit source 19 for passing a substance in transit. In the example shown, it should be observed that only one passage 20 is shown providing communication between the chamber 13 and the transit source 19. In a variant embodiment, the communication passage 20 is formed in the endpiece 4 so as to lead into the blind bore 13₁. Naturally, provision may be made for the transit source 19 to be connected directly to the chamber 13. The transit source 19 may be incorporated in the actuator support 3 or it may be remote from the actuator support 3.

It should be considered that the outer insertion tube 5 is arranged to define directly at least a portion of the internal chamber 13 that communicates exclusively firstly, beside the distal portion 7, with the outside of the outer insertion tube 5, via the orifices 15, and secondly, beside the proximal portion 6, with the transit source 19. The transit source 19 is adapted to inject or deliver a substance to the distal portion 7 so that it escapes via the orifices 15, and/or to suck in or take a substance coming from the distal portion 7 and penetrating into the chamber 13 via the through orifices 15, in order to be taken to the transit source 19.

Naturally, any type of substance may transit via said source 19. For example, provision may be made to deliver a substance from the proximal portion 6 to the outside of the distal portion 7 via the orifices 15, which substance may be in the form of a fluid, a liquid, a gas, a solid material, a light flux, an electromagnetic wave, etc. Similarly, provision may be made to cause a substance to transit from outside the distal portion 7 through the orifices 15 until it reaches the proximal portion, e.g. by means of suction, which substance may be a gas such as smoke, or a liquid (blood, physiological liquid, . . . ).

According to another characteristic of the invention, the instrument 1 includes at least one and generally a series of tubular sheaths or ducts 21ᵢ (where i=1 to n) that are mounted independently of one another inside the outer insertion tube 5 and that pass right through the chamber 13 in order to lead to the outside of the outlet section 8 of the outer insertion tube 5. These tubular ducts 21ᵢ thus pass through the proximal and distal closure systems 10 and 11, extending beside one another and touching or being spaced apart relative to one another. Naturally, the tubular ducts 21ᵢ are flexible or supple in nature since they are mounted inside the insertion tube 5, which is flexible.

In a preferred variant embodiment, the tubular ducts 21ᵢ pass in sealed manner through the proximal and distal closure systems 10 and 11. In this example, the tubular ducts 21ᵢ pass in sealed manner through the transverse wall 4₁ and the closure plug 11. This sealing is achieved in any suitable manner in the closure plug 11 and the transverse wall 4₁. In this preferred variant embodiment, the chamber 13 and the tubular ducts 21ᵢ are sealed relative to one another. Thus, the chamber 13 is sealed relative to each of the other tubular ducts 21ᵢ, and each tubular duct 21ᵢ is itself sealed relative to the chamber 13 and to each of the other tubular ducts 21ᵢ.

It should be considered that each tubular duct 21ᵢ defines internally a channel for passing an associated appliance 22ᵢ (with i=1 to n) that occupies said through channel completely or in part. In general, each appliance 22ᵢ fitted to the instrument 1 depends on the nature of the action(s) to be performed by the endoscope. In non-limiting manner, the appliances 22ᵢ correspond to one or more pieces of equipment, tools, sensors, materials, cables, or accessories. These appliances 22ᵢ may belong to various fields and may, for example, be mechanical, electrical, heat flow, magnetic, chemical, fluid flow, solid, etc., in nature, in order to perform a variety of actions such as, for example, incisions, destructions, sample-taking, measurements, delivery of material (gas, liquid, solid), or delivery of energy (electrical or other).

In the embodiment shown in FIGS. 1 to 4, the instrument 1 has a first tubular duct 21₁ for viewing purposes that includes as its appliance 22₁ an image-forming system mounted in the closure plug 11. This image-forming system 22₁ is connected to a transmission beam extending inside the tubular duct 21₁ and communicating with an image acquisition and processing unit. For example, the image-forming system 22₁ comprises one or more lenses connected to any type of image transmission system.

Similarly, in the embodiment shown in FIGS. 1 to 4, an instrument 1 has a second tubular duct 21₂ referred to as an operator duct, having an appliance 22₂ mounted therein, such as, for example: a sample-taking tool, a fiber, a needle, a brush, a clamp, a probe, etc.

In the embodiment shown in FIG. 5, the instrument includes a third tubular duct 21₃ referred to as a “confinement” duct that includes as its appliance a substance 22₃ associated with an activation system 25 connected to the support 3. The activation system 25 is adapted, on receiving a command, to make the substance 22₃ functional. For example, the substance 22₃ may be medication confined in the tubular duct 21₃ in the form of a packaging cartridge, which cartridge may be opened, for example, under activation from the activation system 25, e.g. in the form of a cable that tears the packaging cartridge, thus releasing the substance that can then escape from the outer insertion tube 5 via the orifices 15. Similarly, the activation system 25 may deliver energy, e.g. electrical, light, heat, or gaseous energy in order to act on one or more substances so as to create a physical and/or chemical reaction in the substance or between a plurality of substances in order to cause the substance(s) to leave the distal end 7 of the outer insertion tube 5.

From the above description, it can be seen that the instrument 1 is suitable for taking on a variety of functions by means of the outer insertion tube 5 and each of the tubular ducts 21ᵢ associated with its respective appliance 22₁ and mounted inside the outer insertion tube 5. Each tubular duct 21ᵢ preferably passes in sealed manner through the transverse wall 4₁ of the endpiece 4, extending inside the insertion tube 5 as far as its distal portion 7, so as to lead to the outside of the outer insertion tube 5 via the outlet section 8 or the orifices 15. It thus appears possible to make use of the entire inside volume of the chamber 13 of the outer insertion tube 5 in order to cause a substance to transit between the outside of the distal end 7 and the inside of the proximal end 6. The inside volume of the chamber 13 corresponds to the inside volume of the outer insertion tube 5 minus each of the individual volumes of the tubular ducts. The inside volume of the chamber 13 corresponds to the volume that is defined between the outsides of the tubular ducts 21ᵢ and that is confined inside the insertion tube 5. Insofar as the entire internal volume of the outer insertion tube 5 is used, it follows that the instrument 1 presents a limited right cross-section while offering a plurality of functions.

It should be observed that the inside volume of the chamber 13 is variable. At least some of the tubular ducts 21ᵢ present the ability to expand or contract, in conjunction with the associated appliances, such that the inside volume of the chamber 13 varies. Similarly, while the instrument 1 is bending and consequently the insertion tube 5 and the tubular ducts 21ᵢ are bending, the inside volume or the distribution of the inside volume of the chamber 13 is caused to vary.

The instrument 1 of the invention is advantageously, but not exclusively, for single use.

FIGS. 6 and 7A, 7B show another variant embodiment of the instrument 1 in accordance with the invention in which the outer insertion tube 5 includes internally an enclosure 30 that is sealed relative to the chamber 13 and that communicates, beside the distal portion 7, with the outside of the tube via at least one additional orifice 31, and, beside the proximal portion 6, with a passage 32 providing communication with an additional transit source 34 for passing a substance in transit. In the example shown, an additional series of orifices 31 pass through the distal portion of the tubular wall 5 in the vicinity of the tubular wall through which the orifices 15 are formed. In this variant embodiment, an internal tube 37 is mounted coaxially inside the outer insertion tube 5 and co-operates therewith to define a space forming the enclosure 30. This internal tube 37 extends to a point set back from the outlet section 8 up to an annular ring 39 that closes the enclosure 30 in sealed manner. Naturally, the ring 39 is located in a position that is set back from the zone of the outer tube 5 in which the orifices 15 are formed. The internal tube 37 is mounted against an abutment wall 40 formed in the support 2 to provide sealing relative to the chamber 13. The enclosure 30 as defined in this way communicates with the additional transit source 34. This additional transit source 34 is adapted to inject or deliver a substance to the distal portion 7 so that it exits via the additional orifices 31, and/or to suck in or take a substance coming from the distal portion 7 and penetrating into the inside of the enclosure 30 via the additional orifices 31 in order to be taken to the additional transit source 34. This additional transit source 34 may be used on its own or in combination with the transit source 19.

In a variant embodiment, at least one tubular tube 21ᵢ is provided with a tearable or ejectable sealing member 45 in the outlet section 8 of the outer tube 5. As can be seen more precisely in FIG. 3A, the tubular duct 21₂ is provided with a sealing member 45 such as a plug or a membrane fastened on the plug 11 so as to close the tubular duct 21₂. This sealing member 45 is torn or ejected by means of the appliance 22₂ exerting mechanical or fluid pressure thereon (FIG. 3D).

FIG. 8 shows a variant embodiment in which the instrument 1 is fitted with at least one sealing membrane 50 closing the orifice(s) 15, and possibly also the additional orifices 31 in the distal portion of the outer insertion tube 5. In the example shown, this sealing membrane 50 covers the inside surface of the tubular wall of the outer insertion tube 5 so as to close the orifices 15 and possibly also the orifices 31. This sealing membrane 50 is tearable by means of fluid pressure or suction or by means of traction or by means of mechanical pressure exerted by a mechanism 51 connected to the support 3. It should be observed that the orifices 15 or indeed 31 may be released of the membrane(s) either successively or simultaneously.

Advantageously, the instrument 1 includes a device enabling the distal portion 7 of the instrument 1 to be folded or deflected relative to the remainder of the external insertion tube. As shown in FIG. 9, the device for folding the distal portion 7 (not shown but made in any suitable known manner) generally comprises one or more actuator mechanisms such as cables fastened to the distal portion 7 of the instrument and connected to an actuator system mounted in the support 3.

From the above description, it can be seen that the instrument 1 is for fitting to an endoscope in the general sense in such a manner as to perform various operations for diagnostic or therapeutic purposes.

The invention presents numerous advantages insofar as it makes it possible to have an instrument 1 of relatively small section while also enabling multiple functions to be provided. For example, the instrument may include an operator tubular duct of relatively large section for passing appliances. Simultaneously, the instrument may also serve, by means of the transit source 19, to perform rinsing, suction, or insufflation of liquid, air, smoke, gas, etc.

The instrument of the invention thus makes it possible for example to take bacteriological samples by injecting physiological serum and then performing suction. Similarly, when a surgical instrument or a laser is used for burning tissue, smoke can be sucked in by the transit source 19 so as to avoid impeding the field of view of the endoscope. Similarly, when inspecting the bronchi, using the orifices 15 enables the patient to be ventilated via the transit source 19.

The invention is not limited to the examples described and shown since various modifications can be applied thereto without going beyond the ambit of the invention.

1. A medical instrument for an endoscope, the instrument comprising a flexible outer insertion tube (5) having a tubular wall possessing a proximal portion (6) for connection to an actuator support (3) and a distal portion (7) presenting an outlet section (8) and connected to a folding mechanism, the instrument being characterized in that:
the outer insertion tube (5) includes closure systems (10, 11) for closing its proximal portion (6) and its distal portion (7) to define an internal chamber (13) communicating beside the distal portion with the outside of the tube via at least one orifice (15) formed through the distal portion of the tubular wall, and beside the proximal portion, with a substance transit source (19); and
a series of flexible tubular ducts (21ᵢ) mounted independently of one another inside the insertion tube (5) passing through the chamber (13) and the closure systems (10, 11) in order to open out to the outside of the outlet section (8) of the distal portion (7) of the tube.

2. A medical instrument for an endoscope according to claim 1, characterized in that the systems (10, 11) for closing the distal and proximal portions (7, 6) are sealed for passing the tubular duct(s) (21ᵢ) in such a manner that the chamber and the tubular duct(s) (21ᵢ) are sealed relative to one another.

3. A medical instrument according to claim 1, characterized in that at least one tubular duct (21ᵢ) defines a through channel for passing an appliance (22ᵢ).

4. A medical instrument for an endoscope according to claim 3, characterized in that at least one tubular duct (21₃) includes as an appliance (22₃), a substance associated with an activation system (25) connected to the actuator support.

5. A medical instrument for an endoscope according to claim 1, characterized in that at least one tubular duct (21₂) is provided in the outlet section of the tube with a tearable or ejectable sealing member (45).

6. A medical instrument for an endoscope according to claim 1, characterized in that it includes an enclosure (30) that is sealed relative to the chamber (13) and that communicates beside the distal portion with the outside of the tube via at least one additional orifice (31) formed in the distal portion of the tubular wall, and beside the proximal portion with a passage (40) communicating with an additional substance transit source (34).

7. A medical instrument for an endoscope according to claim 1, characterized in that it includes at least one sealing membrane (50) closing the orifice(s) (15, 31) formed in the distal portion of the tubular wall, said sealing membrane (50) being tearable by means of fluid pressure or suction or by means of mechanical traction or pressure.

8. An endoscope, characterized in that it includes at least one medical instrument (1) in accordance with claim 1.
